(19) United States
(12) Patent Application Publication
(10) Pub. No.: US 2026/0315500 A1
(43) Pub. Date: Oct. 15, 2026

(54) SOLID CANNABINOID COMPOSITION FOR PARENTERAL USE

(71) Applicant: Bender Analytical Holding B.V., Berg en Dal (NL)

(72) Inventors: Johannes Albertus MICHORIUS, Berg en Dal (NL); Johannes Caspar Mathias Elizabeth BENDER, Berg en Dal (NL)

(73) Assignee: Bender Analytical Holding B.V., Berg en Dal (NL)

(21) Appl. No.: 19/484,315

(22) PCT Filed: Jan. 19, 2024
(86) PCT No.: PCT/EP2024/051266

§ 371 Date: Nov. 13, 2025

(30) Foreign Application Priority Data
May 15, 2023 (EP) 23173362.7
Oct. 12, 2023 (EP) 23203283.9

(51) Int. Cl. A61K 31/00; A61K 9/08; A61K 47/26

(52) U.S. Cl. CPC A61K 31/658; A61K 9/08; A61K 47/26

(57) ABSTRACT

The invention relates to a solid cannabinoid composition comprising:
(i) 0.3-10 wt. % of one or more cannabinoids;
(ii) 4-69 wt. % of one or more non-ionic surfactants with an HLB value of 10-25; and
(iii) at least 20 wt. % of one or more carbohydrates selected from mannitol, sucrose, trehalose and stachyose;
(iv) 0-5 wt. % water;
wherein the combination of the one or more non-ionic surfactants and the one or more carbohydrates constitutes at least 70 wt. % of the composition.

The solid cannabinoid composition of the present invention is highly stable and can suitably be used to prepare a parenterally administrable aqueous liquid within a short period of time, simply by mixing the solid composition with aqueous liquid, such as a sterile isotonic aqueous liquid.

### FIELD OF THE INVENTION

This invention relates to a solid cannabinoid composition for parenteral use. The solid pharmaceutical composition of the present invention can suitably be combined with a sterile aqueous liquid to produce a parenterally administrable liquid.

The solid cannabinoid compositions of the present invention are very stable and dissolve very rapidly in aqueous liquids, thus enabling quick preparation of parenterally administrable liquid.

The present cannabinoid composition can suitably be used in the treatment of neonates at risk of seizures and brain injury, especially in the treatment of Hypoxic-Ischemic Encephalopathy (HIE).

### BACKGROUND OF THE INVENTION

Cannabinoids are highly hydrophobic substances with very poor water solubility. Cannabidiol, for instance, has a water solubility of 0.0627 μg/mL. Tetrahydrocannabinol (THC), another cannabinoid with various therapeutic uses, has a water solubility of 0.0028 mg/ml (23° C.). Thus, in order to prepare a parenterally administrable aqueous liquid containing sufficient cannabinoid to be therapeutically effective, solubility of cannabinoid needs to be enhanced.

Cannabinoids suffer from limited stability, notably as a result of oxidation, especially when applied in water-containing formulations.

HIE is caused by a lack of oxygen and/or blood flow getting to the neonate from the placenta. There are currently no approved drugs or medicinal therapies for HIE and the standard of care is therapeutic hypothermia, which benefits only a select group of new-borns. Each year, more than two million neonates die or evolve to permanent invalidating sequelae worldwide because of HIE.

Preclinical studies offer solid evidence of the neuroprotective effects of Cannabidiol (CBD). Such effects are observable in the short and long term as demonstrated by functional, neuroimaging, histologic and biochemical studies, and are related to the modulation of excitotoxicity, inflammation and oxidative stress—the major components of HIBI pathophysiology. CBD protects neuronal and glial cells, with a remarkable effect on preserving normal myelinogenesis. Therefore CBD is a valuable tool for HIE management since it is safe and effective. It is administered by the parenteral route a posteriori with a broad therapeutic time window.

WO 2018/002636 describes an aqueous parenteral cannabinoid containing formulation comprising:
(i) a cannabinoid;
(ii) an isotonic agent;
(iii) a surfactant; and
(iv) one or more stability enhancers.

WO 2022/174352 concerns a single-phase, injectable cannabinoid formulation for parenteral administration. The formulation contains at least one cannabinoid fully dissolved in a co-solvent system. The co-solvent system contains (i) an effective amount of at least one triglyceride; (ii) an effective amount of at least one C₆ to C₂₂ fatty acid; (iii) from about 1 to about 3% w/v of at least one phosphatidylglycerol and/or salt thereof; and (iv) (optionally) an effective amount of at least one antioxidant (or preservative) to improve shelf-life. The formulations are free of water and ethanol.

WO 2022/017911 concerns the use of cannabidiol (CBD) for the treatment of seizures associated with rare epilepsy syndromes related to brain injury. The described CBD preparation comprises greater than 95% (w/w) CBD and between 0.01 and 0.15% (w/w) tetrahydrocannabinol (THC). An injectable form is not mentioned.

US 2019/015383 describes a water soluble cannabinoid composition comprising:
a purified cannabinoid mixture;
at least one carrier oil; and
at least one water soluble agent chosen from a complex carbohydrate, a polyol, a polysaccharide, an oligosaccharide, or a combination thereof;
   wherein the composition is soluble in water at a temperature less than or equal to about 20° C.; and
   wherein the composition is in the form of agglomerated particles.

US 2023/030491 describes a liquid emulsifying formulation comprising:
i) one or more cannabinoids, and/or a cannabinoid extract;
ii) one or more surfactants; and
iii) one or more oils,
wherein the weight ratio of the one or more surfactants to the one or more cannabinoids, and/or the cannabinoid extract is from about 1:1 to about 20:1 and the weight ratio of the one or more oils to the one or more cannabinoids, and/or the cannabinoid extract is from about 1:1 to about 10:1.

### SUMMARY OF THE INVENTION

The inventors have developed a solid cannabinoid composition that is highly stable and that can suitably be used to prepare a parenterally administrable aqueous liquid within a short period of time, simply by mixing the solid composition with aqueous liquid, such as a sterile isotonic aqueous liquid.

The solid cannabinoid composition according to the present invention comprises:
(i) 0.3-10 wt. % of one or more cannabinoids;
(ii) 4-69 wt. % of one or more non-ionic surfactants with an HLB value of at least 10, preferably of 10-25;
(iii) at least 20 wt. % of one or more carbohydrates selected from mannitol, sucrose, trehalose and stachyose;
(iv) 0-5 wt. % water;
wherein the combination of the one or more non-ionic surfactants and the one or more carbohydrates constitutes at least 70 wt. % of the composition.

The solid cannabinoid composition of the present invention can suitably be prepared by freeze drying an aqueous liquid comprising the one or more cannabinoids, one or more non-ionic surfactants having an HLB of 10 to 25 and one or more carbohydrates selected from mannitol, sucrose, trehalose and stachyose in appropriate concentrations. The solid cannabinoid composition so obtained is highly porous and comprises a carbohydrate matrix that is largely amorphous and in a glassy state. Although the inventors do not wish to be bound by theory, it is believed that this combination of features and the presence of a non-ionic surfactant having a HLB in the range of 10 to 25 contribute to the high stability and the rapid aqueous dissolution of the solid cannabinoid composition of the present invention.

A second aspect of the invention relates to a method of preparing the solid cannabinoid composition of the present invention, said method comprising freeze drying an aqueous liquid comprising:
a. 0.3-10 mg/mL of one or more cannabinoids;
b. one or more non-ionic surfactants with an HLB value of at least 10, preferably of 10-25;
c. one or more carbohydrates selected from mannitol, sucrose, trehalose and stachyose; and
d. at least 85 wt. % water;
wherein the one or more non-ionic surfactants and the one or more cannabinoids are present in the aqueous liquid in a weight ratio of 3:1 to 30:1 and wherein the one or more carbohydrates and the one or more cannabinoids are present in the aqueous liquid in a weight ratio of 3:1 to 300:1.

A third aspect of the invention relates to a vial or a syringe comprising the solid cannabinoid composition according to any one of the preceding claims in an amount that provides 0.2-100 mg cannabinoid.

A fourth aspect of the invention relates to a sterile aqueous liquid for use of in medical treatment, said liquid comprising:
(a) 0.1-10 mg/ml of one or more cannabinoids;
(b) one or more non-ionic surfactants with an HLB value of at least 10, preferably of 10-25;
(c) one or more carbohydrates selected from mannitol, sucrose, trehalose and stachyose; and
(d) at least 85 wt. % water;
wherein the one or more non-ionic surfactants and the one or more cannabinoids are present in the sterile aqueous liquid a weight ratio of 3:1 to 30:1 and wherein the one or more carbohydrates and the one or more cannabinoids are present in the sterile aqueous liquid in a weight ratio of 3:1 to 300:1.

### DETAILED DESCRIPTION OF THE INVENTION

A first aspect of the present invention relates to a solid cannabinoid composition comprising:
(i) 0.3-10 wt. % of one or more cannabinoids;
(ii) 4-69 wt. % of one or more non-ionic surfactants with an HLB value of 10-25;
(iii) at least 20 wt. % of one or more carbohydrates selected from mannitol, sucrose, trehalose and stachyose;
(iv) 0-5 wt. % water;
wherein the combination of the one or more non-ionic surfactants and the one or more carbohydrates constitutes at least 70 wt. % of the composition.

The term “freeze drying” as used herein refers to a process in which a material is dried by cooling it to a temperature below its triple point, the temperature at which the solid, liquid, and gas phases of the material can coexist, followed by sublimation at reduced pressure.

The term “parenteral” as used herein refers to a mode of administration wherein a pharmaceutically active principle is delivered by another route than enteral administration. Examples of parenteral administration include intravenous, intraperitoneal, intramuscular and subcutaneous administration.

The solid cannabinoid composition has preferably been prepared by freeze drying.

The solid cannabinoid composition is preferably provided in the form of a pellet, a powder or a granulate. Most preferably, the composition is provided in the form of a pellet. The pellet preferably has a weight in the range of 50-1,200 mg, more preferably in the range of 100-1,000 mg.

The solid cannabinoid composition provided in the form of a pellet preferably has a density of 30 to 300 mg/mL, more preferably a density of 40 to 200 mg/mL.

The solid cannabinoid composition provided in the form of a powder or a granulate preferably has a density of 10-200 mg/mL, more preferably a density of 20 to 160 mg/L.

The solid cannabinoid composition of the present invention preferably comprises 0.4-8 wt. %, more preferably 0.6-6 wt. % and most preferably 0.8-4 wt. % of the one or more cannabinoids.

The cannabinoids employed in accordance with the present invention may have been isolated from natural materials (e.g. cannabis plant material) or they may have been obtained via synthetic routes. The one or more cannabinoids of the solid composition of the present invention are preferably selected from Tetrahydrocannabinol, Delta-9-tetrahydrocannabinol, Delta-8-Tetrahydrocannabinol, Cannabidiol, Cannabinol, 11-hydroxy-tetrahydrocannabinol, 11-hydroxy-Delta-9-tetrahydrocannabinol, Delta-11-Tetrahydrocannabinol and Tetrahydrocannabivarin, Levonantradol and Nabilone. More preferably, the one or more cannabinoids are selected from Tetrahydrocannabinol, Delta-9-tetrahydrocannabinol, Delta-8-Tetrahydrocannabinol, Cannabidiol and Cannabinol. Even more preferably the one or more cannabinoids are selected from Cannabidiol and Tetrahydrocannabinol. Most preferably the solid cannabinoid composition of the present invention comprises cannabidiol.

The solid cannabinoid composition preferably comprises 5-40 wt. %, more preferably 6-30 wt. %, most preferably 8-24 wt. % of the one or more non-ionic surfactants.

The one or more non-ionic surfactants employed in accordance with the present invention preferably have a HLB in the range of 11 to 20, more preferably in the range of 11 to 18, most preferably in the range of 12 to 16.

According to a preferred embodiment, the one or more non-ionic surfactants are fatty acid esters of polyethylene glycol, more preferably fatty acid esters of polyethylene glycol selected from esters of glycerol polyethylene glycol and C₁₂-C₂₀ hydroxy fatty acids, esters of polyethylene glycol and C₁₂-C₂₀ hydroxy fatty acids, esters of polyethylene glycol sorbitol and C₁₂-C₂₀ fatty acids and combinations thereof.

In one advantageous embodiment, the one or more surfactants are an ester of glycerol polyethylene glycol and C₁₂-C₂₀ hydroxy fatty acids, more preferably C₁₆-C₁₈ hydroxy fatty acids. Most preferably, the non-ionic surfactant is an ester of glycerol polyethylene glycol and ricinoleic acid. Kolliphor® ELP is a commercially available example of such a surfactant.

In another advantageous embodiment, the one or more surfactants are an ester polyethylene glycol and C₁₂-C₂₀ hydroxy fatty acids, more preferably C₁₆-C₁₈ hydroxy fatty acids. Most preferably, the non-ionic surfactant is an ester of polyethylene glycol and 12-hydroxy stearic acid. Kolliphor® HS 15 is a commercially available example of such a surfactant.

According to another advantageous embodiment, the one or more surfactants are esters of polyethylene glycol sorbitol and C₁₂-C₂₀ fatty acids, more preferably C₁₆-C₁₈ fatty acids. Most preferably, the non-ionic surfactant is an ester of polyethylene glycol sorbitol and oleic acid. Polysorbate 80 is an example of such a surfactant.

The solid cannabinoid composition preferably comprises at least 30 wt. %, more preferably 40-95 wt. %, even more preferably 60-93 wt. % and most preferably 75-92 wt. % of the one or more carbohydrates.

The one or more carbohydrates are preferably selected from mannitol and sucrose. Most preferably, the carbohydrate employed is mannitol.

According to a particularly preferred embodiment, the solid cannabinoid composition contains at least 80 wt. % of the one or more carbohydrates in an amorphous state.

The combination of the one or more non-ionic surfactants and the one or more carbohydrates constitutes at least 80 wt. %, more preferably at least 85 wt. % of the solid cannabinoid composition.

Preferably, the combination of the one or more cannabinoids, the one or more non-ionic surfactants and the one or more carbohydrates constitutes at least 75 wt. %, more preferably at least 85 wt. % and most preferably at least 90 wt. % of the solid cannabinoid composition.

The one or more non-ionic surfactants and the one or more cannabinoids are preferably present in the solid cannabinoid composition in a weight ratio of 3:1 to 30:1, more preferably in a weight ratio of 4:1 to 25:1, even more preferably in a weight ratio of 5:1 to 20:1.

Preferably, the one or more carbohydrates and the one or more cannabinoids are present in the solid cannabinoid composition in a weight ratio of 3:1 to 300:1, more preferably in a weight ratio of 15:1 to 200:1, even more preferably of 20:1 to 150:1, most preferably of 30:1 to 120:1.

The solid cannabinoid composition of the present invention preferably has a reconstitution time of less than 180 seconds, more preferably of less than 120 second and most preferably of less than 60 seconds as determined by the impedance-based method described by Werk et al. (An Impedance-Based Method to Determine Reconstitution Time for Freeze-Dried Pharmaceuticals, J. of Pharm. Sc. (2015) 104:2948-2955), using 300 mg of the solid cannabinoid composition and 5 ml of water for injection having a temperature of 20° C.

Besides rapidly dissolving, the solid cannabinoid composition of the present invention preferably forms a clear solution. Thus, it can easily be established by medical staff if the solid composition is adequately dissolved and ready for parenteral administration.

According to a preferred embodiment, reconstitution of 300 mg of the solid cannabinoid composition of the present invention with 5 ml of water for injection having a temperature of 20° C. yields a clear solution. The reconstituted composition is considered clear if its clarity is the same as that of water R or if its opalescence does not exceed. that of reference suspension I as per Table 2.2.1.-2, Chapter 2.2.1. CLARITY AND DEGREE OF OPALESCENCE OF LIQUIDS, European Pharmacopoeia.

Besides the one or more cannabinoids, the solid cannabinoid composition preferably does not contain significant amounts of lipophilic components as the presence of such components in the composition may result in the formation of turbid or hazy emulsions when the solid composition is reconstituted in aqueous liquid for parenteral use.

Accordingly, in a preferred embodiment, the solid cannabinoid composition contains 0-1 wt. % triglycerides, more preferably 0-0.5 wt. % triglycerides, even more preferably 0-0.3 wt. % triglycerides and most preferably 0-0.1 wt. % triglycerides.

Preferably, the solid cannabinoid composition contains 0-1 wt. % of oil, more preferably 0-0.5 wt. % of oil, even more preferably 0-0.3 wt. % of oil and most preferably 0-0.1 wt. % of oil, said oil being selected from fatty acids, monoglycerides, diglycerides, triglycerides, propylene glycol mono esters, propylene glycol diesters and combinations thereof.

The solid cannabinoid composition of the present invention preferably contains triglycerides and the one or more cannabinoids in a weight ratio of triglycerides:one or more cannabinoids that is in the range of 0 to 1, more preferably in the range of 0 to 0.3, even more preferably in the range of 0 to 0.1 and most preferably in the range of 0 to 0.03.

Oil and the one or more cannabinoids are preferably present in the solid cannabinoid composition in a weight ratio oil:one or more cannabinoids that is in the range of 0 to 1, more preferably in the range of 0 to 0.3, even more preferably in the range of 0 to 0.1 and most preferably in the range of 0 to 0.03, said oil being selected from fatty acids, monoglycerides, diglycerides, triglycerides, propylene glycol mono esters, propylene glycol diesters and combinations thereof.

The water content of the solid cannabinoid composition is preferably in the range of 0-3 wt. %, more preferably in the range of 0-2.5 wt. %, most preferably in the range of 0-2 wt. %.

Besides cannabinoid, non-ionic surfactant, carbohydrate and water, the solid cannabinoid composition of the present invention may suitably contain additional ingredients. Examples of such ingredients include: salts, polymers (such as polyvinylpyrrolidone), anti-oxidants (e.g. ascorbic acid) and isotonic agents (e.g. glycerol).

According to a particularly preferred embodiment, the solid cannabinoid composition, when dispersed in distilled water having a temperature of 37° C. in a concentration of 10 to 200 mg/mL, yields an aqueous liquid having an osmolarity in the range of 100 to 800 mOsm/L, more preferably in the range of 150 to 500 mOsm/L, most preferably in the range of 200 to 400 mOsm/L.

According to a second aspect of the present invention there is provided a method of preparing the solid cannabinoid composition of the present invention. The method comprises freeze drying an aqueous liquid comprising:
(a) 0.1-10 mg/ml of one or more cannabinoids;
(b) one or more non-ionic surfactants with an HLB value of 10-25;
(c) one or more carbohydrates selected from mannitol, sucrose, trehalose and stachyose; and
(d) at least 85 wt. % water;
wherein the one or more non-ionic surfactants and the one or more cannabinoids are present in the aqueous liquid in a weight ratio of 3:1 to 30:1 and wherein the one or more carbohydrates and the one or more cannabinoids are present in the aqueous liquid in a weight ratio of 3:1 to 300:1.

The one or more non-ionic surfactants and the one or more cannabinoids are preferably present in the aqueous liquid in a weight ratio of 4:1 to 25:1, more preferably in the a weight ratio of 5:1 to 20:1.

Preferably, the one or more carbohydrates and the one or more cannabinoids are present in the aqueous liquid in a weight ratio of 15:1 to 200:1, more preferably of 20:1 to 150:1, most preferably of 30:1 to 120:1.

Preferably, the aqueous liquid that is freeze dried in the present method comprises 0.2-8 mg/ml of one or more cannabinoids, more preferably 0.4-4 mg/ml of one or more cannabinoids.

Freeze drying of the aqueous liquid comprises the following three successive phase: (1) freezing; (2) primary drying (sublimation); (3) secondary drying (absorption).

The freeze drying of the aqueous liquid may comprise additional process steps, such as one or more annealing steps.

In the primary drying phase the pressure is lowered and heat is added to the material in order for the water to sublimate. Most of the water in the material is removed in this phase. In the secondary drying phase ionically-bound water molecules are removed. By raising the temperature higher than in the primary drying phase, the bonds are broken between the material and the water molecules. Once the freeze drying process is complete, the vacuum is broken.

After freeze drying, the solid cannabinoid composition is preferably introduced into a vial or syringe. According to a particularly preferred embodiment, the solid cannabinoid composition is introduced and sealed into the vial or the syringe under reduced pressure. The inventors have found that this embodiment offers the important advantage that when the solid cannabinoid composition is combined with aqueous solvent, foaming is effectively minimised. In a preferred embodiment, the solid cannabinoid composition is introduced and sealed into the vial or the syringe at a pressure of less than 10 kPa, more preferably of less than 5 kPa and most preferably of 0-1 kPa.

A third aspect of the present invention relates to a vial or a syringe comprising the solid cannabinoid composition of the present invention in an amount providing 0.2-100 mg, more preferably 0.5-40 mg, even more preferably 1-20 mg of cannabinoid and most preferably 2-10 mg of cannabinoid.

In a preferred embodiment, the syringe comprises two chambers, a first chamber containing the solid cannabinoid composition and a second chamber containing a sterile isotonic aqueous solvent. Preferably, the syringe is configured to allow the contents of the two chamber to be mixed to produce an aqueous cannabinoid solution and to allow expulsion of the aqueous cannabinoid solution from the syringe, e.g. by pushing a syringe plunger.

As already explained above, the vial or the chamber of the syringe that holds the solid cannabinoid composition is preferably under reduced pressure. Preferably, the reduced pressure is at most 50 kPa, more preferably at most 30 kPa, most preferably 0.0001 to 10 kPa. A fourth aspect of the present invention relates to a sterile aqueous liquid for use in medical treatment, said liquid comprising:
(a) 0.1-10 mg/ml of one or more cannabinoids;
(b) one or more non-ionic surfactants with an HLB value of 10-25;
(c) one or more carbohydrates selected from mannitol, sucrose, trehalose and stachyose; and
(d) at least 85 wt. % water;
wherein the one or more non-ionic surfactants and the one or more cannabinoids are present in the sterile aqueous liquid in a weight ratio of 3:1 to 30:1 and wherein the one or more carbohydrates and the one or more cannabinoids are present in the sterile aqueous liquid in a weight ratio of 3:1 to 300:1.

According to a particularly preferred embodiment, the sterile aqueous liquid can be obtained by dispersing the solid cannabinoid composition of the present invention into water for injection.

Preferably, the sterile aqueous liquid used in the treatment comprises 0.2-8 mg/mL of the one or more cannabinoids, more preferably 0.4-4 mg/ml of the one or more cannabinoids.

The one or more cannabinoids in the sterile aqueous liquid are preferably selected from Tetrahydrocannabinol, Delta-9-tetrahydrocannabinol, Delta-8-Tetrahydrocannabinol, Cannabidiol, Cannabinol, 11-hydroxy-tetrahydrocannabinol, 11-hydroxy-Delta-9-tetrahydrocannabinol, Delta-11-Tetrahydrocannabinol and Tetrahydrocannabivarin, Levonantradol and Nabilone. More preferably, the one or more cannabinoids are selected from Tetrahydrocannabinol, Delta-9-tetrahydrocannabinol, Delta-8-Tetrahydrocannabinol, Cannabidiol and Cannabinol. Even more preferably, the one or more cannabinoids are selected from Cannabidiol and Tetrahydrocannabinol. Most preferably the cannabinoids is cannabidiol.

The one or more non-ionic surfactants employed preferably have a HLB in the range of 11 to 20, more preferably in the range of 11 to 18, most preferably in the range of 12 to 16.

The one or more non-ionic surfactants of the sterile aqueous liquid are preferably fatty acid esters of polyethylene glycol, more preferably fatty acid esters of polyethylene glycol selected from esters of glycerol polyethylene glycol and C₁₂-C₂₀ hydroxy fatty acids, esters polyethylene glycol and C₁₂-C₂₀ hydroxy fatty acids, esters of polyethylene glycol sorbitol and C₁₂-C₂₀ fatty acids and combinations thereof.

In an advantageous embodiment, the one or more surfactants are an ester of glycerol polyethylene glycol and C₁₂-C₂₀ hydroxy fatty acids. Most preferably, the non-ionic surfactant is an ester of glycerol polyethylene glycol and ricinoleic acid. Kolliphor® ELP is a commercially available example of such a surfactant.

In another advantageous embodiment, the one or more surfactants are an ester polyethylene glycol and C₁₂-C₂₀ hydroxy fatty acids. Most preferably, the non-ionic surfactant is an ester of polyethylene glycol and 12-hydroxy stearic acid. Kolliphor® HS 15 is a commercially available example of such a surfactant.

According to another advantageous embodiment, the one or more surfactants are esters of polyethylene glycol sorbitol and C₁₂-C₂₀ fatty acids. Most preferably, the non-ionic surfactant is an ester of polyethylene glycol sorbitol and oleic acid. Polysorbate 80 is an example of such a surfactant.

Preferably, the one or more carbohydrates are selected from mannitol, sucrose and combinations thereof. Most preferably, the carbohydrate employed is mannitol.

The one or more non-ionic surfactants and the one or more cannabinoids are preferably present in the sterile aqueous liquid in a weight ratio of 4:1 to 25:1, more preferably in the a weight ratio of 5:1 to 20:1.

Preferably, the one or more carbohydrates and the one or more cannabinoids are present in the sterile aqueous liquid in a weight ratio of 15:1 to 200:1, more preferably of 20:1 to 150:1 most preferably of 30:1 to 120:1.

The sterile aqueous liquid preferably contains 0-10 mg/L triglycerides, more preferably 0-6 mg/L triglycerides, even more preferably 0-3 mg/L triglycerides and most preferably 0-1 mg/L triglycerides.

Oil is preferably contained in the sterile aqueous liquid in a concentration of 0-10 mg/L, more preferably of 0-6 mg/L, even more preferably of 0-3 mg/L and most preferably of 0-1 mg/L, said oil being selected from fatty acids, monoglycerides, diglycerides, triglycerides, propylene glycol mono esters, propylene glycol diesters and combinations thereof.

The sterile aqueous liquid preferably contains triglycerides and the one or more cannabinoids in a weight ratio of triglycerides:one or more cannabinoids that is in the range of 0 to 1, more preferably in the range of 0 to 0.3, even more preferably in the range of 0 to 0.1 and most preferably in the range of 0 to 0.03.

Oil and the one or more cannabinoids are preferably contained in the sterile aqueous liquid in a weight ratio of oil:one or more cannabinoids that is in the range of 0 to 1, more preferably in the range of 0 to 0.3, even more preferably in the range of 0 to 0.1 and most preferably in the range of 0 to 0.03, said oil being selected from fatty acids, monoglycerides, diglycerides, triglycerides, propylene glycol mono esters, propylene glycol diesters and combinations thereof.

According to a particularly preferred embodiment, said aqueous liquid has a pH of 2-11, more preferably a pH of 4-9, most preferably a pH of 6.5-8.0.

In accordance with a particularly preferred embodiment, the aforementioned aqueous liquid has an osmolarity in the range of 100 to 800 mOsm/L, more preferably in the range of 180 to 500 mOsm/L, most preferably in the range of 200 to 400 mOsm/L.

According to a particularly preferred embodiment, the sterile aqueous liquid is prepared by combining the solid cannabinoid composition of the present invention with a sterile liquid. Preferably, the sterile liquid that is used in preparation of the sterile aqueous liquid contains at least 90 wt. % water, more preferably at least 95 wt. % water.

In accordance with a particularly preferred embodiment, the medical treatment comprises parenteral administration, more preferably intravenous, intraperitoneal, intramuscular or subcutaneous administration, most preferably intravenous administration of the sterile aqueous liquid.

The medical treatment of the present invention preferably comprises administration of the sterile aqueous liquid to a neonate. Preferably, the sterile aqueous liquid is administered to the neonate to provide cannabinoid in a dosage of 1-15 mg, more preferably of 2-10 mg.

In a particularly preferred embodiment, the present medical treatment is the treatment of neonates at risk of seizures and brain injury, especially the treatment of Hypoxic-ischemic Encephalopathy (HIE).

The invention is further illustrated by the following non-limiting examples.

### EXAMPLES

### Example 1

Aqueous solutions containing cannabidiol were prepared on the basis of the recipes shown in Table 1.

TABLE 1

| | Wt. % | | |
|---|---|---|---|
| | 1 | 2 | 3 |
| Cannabidiol | 0.1 | 0.1 | 0.1 |
| Kolliphor ELP ¹ | 1.0 | 1.0 | 1.0 |
| Mannitol | 1.0 | 5.0 | 10.0 |
| Demi-water | 97.9 | 93.9 | 88.9 |

¹ Ester of glycerol polyethylene glycol and ricinoleic acid (HLB 12-14)

Cannabidiol was first dissolved in the surfactant and mannitol was first dissolved in demi-water before mixing the two solutions using a magnetic stirrer.

5 ml aliquots of the cannabidiol solutions were filled into clear glass tubular injection vials with crimp neck (8R: diameter=22 mm, height=45 mm), frozen and lyophilised.

Lyophilisation was carried out in a lyophiliser (Drawell Analytical, DW-10N series), using a vacuum pump (2XZ-2 rotary vane vacuum pump (A4218)).

The lyophilisation run settings were as follows:
cold trap temperature −60° C.,
pressure 1.0-3.0 Pa,
duration +/−24 hours.

The glass vials containing the lyophilised pellets were sealed by closing the vials with a screw cap comprising a septum.

The dissolution rate (reconstitution time) of lyophilised cannabidiol pellets was assessed within the clear glass vials by introducing 5 mL of demi-water having a temperature of 20° C. through the septum into the vial with a syringe, gently shaking the container manually, and determining the time passed until a clear solution was formed. The results are summarised in Table 2.

TABLE 2

| | 1 | 2 | 3 |
|---|---|---|---|
| Dissolution time (seconds) | 31 | 36 | 64 |

Cannabinoid pellets 2 offer the advantage that they can easily be diluted with water for injection to produce an isotonic intravenous solution that can suitably be administered to neonates by injection to provide a dose of 2-10 mg cannabinoid.

### Example 2

Example 1 was repeated starting from formulation number 3, except that mannitol was replaced by sucrose. This time it took slightly more than 2 minutes to achieve complete dissolution of the pellet.

### Example 3

Example 1 was repeated starting from formulation number 3, except that vial was lyophilized and sealed at a pressure of 2 Pa. The dissolution test gave a similar dissolution time as in Example 1, but substantially less foaming was observed during shaking.

### Example 4

Aqueous solutions containing cannabidiol were prepared on the basis of the recipes shown in Table 3.

TABLE 3

| | Wt. % | | | | | | | |
|---|---|---|---|---|---|---|---|---|
| | 1 | 2 | 3 | 4 | 5 | 6 | 7 | 8 |
| Cannabidiol | 0.05 | 0.1 | 0.1 | 0.05 | 0.1 | 0.1 | 0.05 | 0.1 |
| Kolliphor ELP ¹ | 0.5 | 1 | 5 | | | | | |
| Kolliphor HS15 ² | | | | 0.5 | 1 | 5 | | |
| Polysorbate 80 ³ | | | | | | | 1 | 5 |
| Mannitol | 5 | 5 | 5 | 5 | 5 | 5 | 5 | 5 |

| | |
|---|---|
| Demi-water | remainder |

¹ Ester of glycerol polyethylene glycol and ricinoleic acid (HLB 12-14)
² Ester of polyethylene glycol and 12-hydroxy stearic acid (HLB 14-16)
³ Ester of polyethylene glycol sorbitol and oleic acid (HLB 15)

Cannabidiol was first dissolved in the surfactant and mannitol was first dissolved in demi-water before mixing the two solutions using a magnetic stirrer. All the cannabidiol solutions so obtained were completely clear, indicating that after lyophilisation, the freeze dried material will form a clear solution upon reconstitution with water.

### Example 5

Aqueous solutions containing cannabidiol were prepared on the basis of the recipes shown in Table 4.

TABLE 4

| | Wt. % | | | | | | | |
|---|---|---|---|---|---|---|---|---|
| | 1 | 2 | 3 | A | B | C | D | E |
| Cannabidiol | 0.1 | 0.1 | 0.1 | 0.1 | 0.1 | 0.1 | 0.1 | 0.1 |
| Kolliphor ELP ¹ | 1 | 1 | 1 | 1 | 1 | 1 | 1 | 1 |
| Mannitol | 5 | | | | | | | |
| Trehalose | | 5 | | | | | | |
| Stachyose | | | 5 | | | | | |
| Maltose | | | | 5 | | | | |
| Fructose | | | | | 5 | | | |
| Glucose | | | | | | 5 | | |
| Sorbitol | | | | | | | 5 | |
| Xylitol | | | | | | | | 5 |

| | |
|---|---|
| Demi-water | Remainder |

¹ Ester of glycerol polyethylene glycol and ricinoleic acid (HLB 12-14)

Cannabidiol was first dissolved in the surfactant and each of the carbohydrates was first dissolved in demi-water before mixing the two solutions using a magnetic stirrer.

5 ml aliquots of the cannabidiol solutions so obtained were filled into clear glass tubular injection vials with crimp neck (8R: diameter=22 mm, height=45 mm), frozen and lyophilised.

Lyophilisation was carried out in a lyophiliser (Drawell Analytical, DW-10N series), using a vacuum pump (2XZ-2 rotary vane vacuum pump (A4218)).

The lyophilisation run settings were as follows:
cold trap temperature −60° C.,
pressure 1.0-3.0 Pa,
duration +/−24 hours.

The glass vials containing the lyophilised pellets were sealed by closing the vials with a screw cap comprising a septum.

The dissolution rate of lyophilised cannabidiol pellets was assessed within the clear glass vials by introducing 5 mL of demi-water having a temperature of 20° C. through the septum into the vial with a syringe, gently shaking the container manually, and determining the time passed until a clear solution was formed or until the moment no further dissolution occurred. In addition, also the appearance of lyophilisate and of the reconstituted liquids was evaluated. The results obtained for the lyophilised cannabidiol solutions of Table 4 are summarised in Table 5.

TABLE 5

| | Lyophilised cannabidiol solution | | | | | | | |
|---|---|---|---|---|---|---|---|---|
| | 1 | 2 | 3 | A | B | C | D | E |
| Dissolution | 42 | 43 | 32 | 23 | >150 | 117 | 21 | >150 |
| time (sec.) | | | | | | | | |
| Appearance of | + | + | + | + | −* | −* | + | −* |
| lyophilisate | | | | | | | | |
| Appearance | Clear | clear | clear | hazy | clear | hazy | hazy | hazy |
| liquid | | | | | | | | |

*Cake collapse and/or melt back occurred in these conditions, resulting in a lyophilizate that cannot be reliably reconstituted

### Example 6

Aqueous solutions containing cannabidiol were prepared on the basis of the recipes shown in Table 6.

TABLE 6

| | Wt. % | |
|---|---|---|
| | 1 | 2 |
| Cannabidiol | 0.1 | 0.1 |
| Kolliphor ELP ¹ | 1.0 | 1.0 |
| Trehalose | 1.0 | 10.0 |
| Demi-water | 97.9 | 88.9 |

¹ Ester of glycerol polyethylene glycol and ricinoleic acid (HLB 12-14)

Cannabidiol was first dissolved in the emulsifier and trehalose was first dissolved in demi-water before mixing the two solutions using a magnetic stirrer.

The dissolution rate of lyophilised cannabidiol pellets, the appearance of the lyophilizates and of the reconstituted liquids were evaluated in the same way as in Example 5. The results are summarised in Table 7.

TABLE 7

| | 1 | 2 |
|---|---|---|
| Dissolution time (seconds | 42 | 41 |
| Appearance of lyophilisate | + | + |
| Appearance liquid | clear | clear |

### Example 7

Aqueous solutions containing cannabidiol were prepared on the basis of the recipes shown in Table 8.

TABLE 8

| | Wt. % | |
|---|---|---|
| | 1 | 2 |
| Cannabidiol | 0.1 | 0.1 |
| Kolliphor ELP ¹ | 1.0 | 1.0 |
| Stachyose | 1.0 | 10.0 |
| Demi-water | 97.9 | 88.9 |

¹ Ester of glycerol polyethylene glycol and ricinoleic acid (HLB 12-14)

Cannabidiol was first dissolved in the emulsifier and stachyose was first dissolved in demi-water before mixing the two solutions using a magnetic stirrer.

The dissolution rate of lyophilised cannabidiol pellets, the appearance of the lyophilizates and of the reconstituted liquids were evaluated in the same way as in Example 5. The results are summarised in Table 9.

TABLE 9

| | 1 | 2 |
|---|---|---|
| Dissolution time (seconds) | 25 | 22 |
| Appearance of lyophilisate | + | + |
| Appearance liquid | clear | clear |

### Example 8

Aqueous solutions containing tetrahydrocannabinol were prepared on the basis of the recipes shown in Table 10.

TABLE 10

| | Wt. % | | | | | | | | | |
|---|---|---|---|---|---|---|---|---|---|---|
| | 1 | 2 | 3 | 4 | 5 | 6 | 7 | 8 | 9 | 10 |
| Tetrahydro- | 0.05 | 0.1 | 0.1 | 0.5 | 0.05 | 0.1 | 0.1 | 0.5 | 0.05 | 0.1 |
| cannabinol | | | | | | | | | | |
| Kolliphor ELP ¹ | 0.5 | 1 | 5 | 5 | | | | | | |
| Kolliphor HS15 ² | | | | | 0.5 | 1 | 5 | 5 | | |
| Polysorbate 80 ³ | | | | | | | | | 1 | 5 |
| Mannitol | 5 | 5 | 5 | 5 | 5 | 5 | 5 | 5 | 5 | 5 |

Remainder
¹ Ester of glycerol polyethylene glycol and ricinoleic acid (HLB 12-14)
² Ester of polyethylene glycol and 12-hydroxy stearic acid (HLB 14-16)
³ Ester of polyethylene glycol sorbitol and oleic acid (HLB 15)

Tetrahydrocannabinol was first dissolved in the emulsifier and mannitol was first dissolved in demi-water before mixing the two solutions using a magnetic stirrer.

All the tetrahydrocannabinol solutions so obtained were completely clear, indicating that after lyophilisation, the dry material will form a clear solution upon reconstitution with water.

### Example 9

Tetrahydrocannabinol solution 2 of Example 7 was lyophilised in the same way as described in Example 5.

The dissolution rate of lyophilizate, and the appearance of lyophilisate and of the reconstituted liquids were evaluated in the same way as in Example 5. It was found that the lyophilizate formed a clear solution in 23 seconds.

### Comparative Example A

The mixture of SNEDD, water and sorbitol of Example 11 of US 2023/0030491 was reproduced. The aqueous liquid so obtained was hazy, indicating that after lyophilisation, the dry material will also form a hazy liquid upon reconstitution with water.

### Comparative Example B

Aqueous solutions containing cannabidiol were prepared on the basis of the recipes shown in Table 11.

TABLE 11

| | Wt. % | | |
|---|---|---|---|
| | A | B | C |
| Cannabidiol | 0.05 | 0.1 | 0.1 |
| Kolliphor 188 ¹ | 0.5 | 1 | 5 |
| Mannitol | 5 | 5 | 5 |

| | |
|---|---|
| Demi-water | Remainder |

¹ Block co-polymer of ethylene oxide and propylene oxide (HLB 29)

All the cannabidiol formulations obtained were hazy dispersions.

1. A solid cannabinoid composition comprising:
(i) 0.3-10 wt. % of one or more cannabinoids;
(ii) 4-69 wt. % of one or more non-ionic surfactants with an HLB value of 10-25;
(iii) at least 20 wt. % of one or more carbohydrates selected from mannitol, sucrose, trehalose and stachyose;
(iv) 0-5 wt. % water;
wherein the combination of the one or more non-ionic surfactants and the one or more carbohydrates constitutes at least 70 wt. % of the composition.

2. The solid cannabinoid composition according to claim 1, wherein the solid composition has a density of 10 to 200 mg/mL.

3. The solid cannabinoid composition according to claim 1 or 2, wherein the one or more cannabinoids are selected from Tetrahydrocannabinol, Delta-9-tetrahydrocannabinol, Delta-8-Tetrahydrocannabinol, Cannabidiol, Cannabinol, 11-hydroxy-tetrahydrocannabinol, 11-hydroxy-Delta-9-tetrahydrocannabinol, Delta-11-Tetrahydrocannabinol and Tetrahydrocannabivarin, Levonantradol and Nabilone, preferably selected from Tetrahydrocannabinol, Delta-9-tetrahydrocannabinol, Delta-8-Tetrahydrocannabinol, Cannabidiol and Cannabinol, most preferably selected from Cannabidiol and Tetrahydrocannabinol.

4. The solid cannabinoid composition according to claim 3, wherein the cannabinoid is selected from cannabidiol, tetrahydrocannabinol and combinations thereof.

5. The solid cannabinoid composition according to any one of the preceding claims, wherein the composition contains at least 4 wt. % of non-ionic surfactants with an HLB value in the range of 11 to 18, preferably in the range of 12 to 16.

6. The solid cannabinoid composition according to any one of the preceding claims, wherein the one or more non-ionic surfactants are fatty acid esters of polyethylene glycol.

7. The solid cannabinoid composition according to claim 6, wherein the fatty acid esters of polyethylene glycol are selected from esters of glycerol polyethylene glycol and C₁₂-C₂₀ hydroxy fatty acids, esters of polyethylene glycol and C₁₂-C₂₀ hydroxy fatty acids, esters of polyethylene glycol sorbitol and C₁₂-C₂₀ fatty acids and combinations thereof.

8. The solid cannabinoid composition according to any one of the preceding claims, wherein the one or more carbohydrates are selected from mannitol, sucrose, trehalose and stachyose.

9. The solid cannabinoid composition according to claim 8, wherein the one or more carbohydrates is mannitol.

10. The solid cannabinoid composition according to any one of the preceding claims, wherein the solid cannabinoid composition contains 0-1 wt. % triglycerides.

11. The solid cannabinoid composition according to claim 10, wherein the solid cannabinoid composition contains 0-1 wt. % of oil, said oil being selected from fatty acids, monoglycerides, diglycerides, triglycerides, propylene glycol mono esters, propylene glycol diesters and combinations thereof.

12. The solid cannabinoid composition according to any one of the preceding claims, wherein the solid cannabinoid composition contains triglycerides and the one or more cannabinoids in a weight ratio of triglycerides:one or more cannabinoids that is in the range of 0 to 1.

13. The solid cannabinoid composition according to claim 12, wherein the solid cannabinoid composition contains oil and the one or more cannabinoids in a weight ratio of oil:one or more cannabinoids that is in the range of 0 to 1, said oil being selected from fatty acids, monoglycerides, diglycerides, triglycerides, propylene glycol mono esters, propylene glycol diesters and combinations thereof.

14. The solid cannabinoid composition according to any one of the preceding claims, wherein the composition, when dispersed in distilled water having a temperature of 37° C. in a concentration of 10 to 200 mg/mL, yields an aqueous liquid having an osmolarity in the range of 100 to 800 mOsm/L.

15. A method of preparing the solid cannabinoid composition according to any one of claims 1-14, said method comprising freeze drying an aqueous liquid comprising:
a. 0.1-10 mg/ml of one or more cannabinoids;
b. one or more non-ionic surfactants with an HLB value of 10-25;
c. one or more carbohydrates selected from mannitol, sucrose, trehalose and stachyose; and
d. at least 85 wt. % water;
wherein the one or more non-ionic surfactants and the one or more cannabinoids are present in the aqueous liquid in a weight ratio of 3:1 to 30:1 and wherein the one or more carbohydrates and the one or more cannabinoids are present in the aqueous liquid in a weight ratio of 3:1 to 300:1.

16. A vial or a syringe comprising the solid cannabinoid composition according to any one of claims 1-14 in an amount that provides 0.2-100 mg cannabinoid.

17. A sterile aqueous liquid for use in medical treatment, said liquid comprising:
(a) 0.1-10 mg/ml of one or more cannabinoids;
(b) one or more non-ionic surfactants with an HLB value of 10-25;
(c) one or more carbohydrates selected from mannitol, sucrose, trehalose and stachyose; and
(d) at least 85 wt. % water;
wherein the one or more non-ionic surfactants and the one or more cannabinoids are present in the sterile aqueous liquid in a weight ratio of 3:1 to 30:1 and wherein the one or more carbohydrates and the one or more cannabinoids are present in the sterile aqueous liquid in a weight ratio of 3:1 to 300:1.

18. The sterile aqueous liquid for use in medical treatment according to claim 17, wherein the one or more cannabinoids are selected from Tetrahydrocannabinol, Delta-9-tetrahydrocannabinol, Delta-8-Tetrahydrocannabinol, Cannabidiol, Cannabinol, 11-hydroxy-tetrahydrocannabinol, 11-hydroxy-Delta-9-tetrahydrocannabinol, Delta-11-Tetrahydrocannabinol and Tetrahydrocannabivarin, Levonantradol and Nabilone, preferably selected from Tetrahydrocannabinol, Delta-9-tetrahydrocannabinol, Delta-8-Tetrahydrocannabinol, Cannabidiol and Cannabinol, most preferably selected from Cannabidiol and Tetrahydrocannabinol.

19. The sterile aqueous liquid for use in medical treatment according to claim 17 or 18, wherein the one or more non-ionic surfactants have an HLB value in the range of 11 to 18, preferably in the range of 12 to 16.

20. The sterile aqueous liquid for use in medical treatment according to claim according to any one of claims 17-19, wherein the one or more non-ionic surfactants are fatty acid esters of polyethylene glycol.

21. The sterile aqueous liquid for use in medical treatment according to any one of claims 17-20, wherein the one or more non-ionic surfactants are selected from esters of glycerol polyethylene glycol and C₁₂-C₂₀ hydroxy fatty acids, esters polyethylene glycol and C₁₂-C₂₀ hydroxy fatty acids, esters of polyethylene glycol sorbitol and C₁₂-C₂₀ fatty acids and combinations thereof.

22. The sterile aqueous liquid for use in medical treatment according to any one of claims 17-21, wherein the one or more carbohydrates are selected from mannitol, sucrose, trehalose and stachyose.

23. The sterile aqueous liquid for use in medical treatment according to claim 22, wherein the one or more carbohydrates are mannitol.

24. The sterile aqueous liquid for use in medical treatment according to any one of claims 17-23, wherein the sterile aqueous liquid contains 0-10 mg/L triglycerides.

25. The sterile aqueous liquid for use in medical treatment according to claim 24, wherein the sterile aqueous liquid contains 0-10 mg/L of oil, said oil being selected from fatty acids, monoglycerides, diglycerides, triglycerides, propylene glycol mono esters, propylene glycol diesters and combinations thereof.

26. The sterile aqueous liquid for use in medical treatment according to any one of claims 17-25, wherein the sterile aqueous liquid contains triglycerides and the one or more cannabinoids in a weight ratio of triglycerides:one or more cannabinoids that is in the range of 0 to 1.

27. The sterile aqueous liquid for use in medical treatment according to claim 26, wherein the sterile aqueous liquid contains oil and the one or more cannabinoids in a weight ratio of oil:one or more cannabinoids that is in the range of 0 to 1, said oil being selected from fatty acids, monoglycerides, diglycerides, triglycerides, propylene glycol mono esters, propylene glycol diesters and combinations thereof.

28. The sterile aqueous liquid for use in medical treatment according to any one of claims 17-27, wherein the sterile aqueous liquid is prepared by combining a solid cannabinoid composition according to any one of claims 1-14 with a sterile liquid containing at least 90 wt. % water.

29. The sterile aqueous liquid for use in medical treatment according to any one of claims 17-28, wherein the medical treatment comprises parenteral administration, preferably intravenous administration of the sterile aqueous liquid.
